(19) United States
(12) Patent Application Publication
(10) Pub. No.: US 2008/0234680 A1
(43) Pub. Date: Sep. 25, 2008

(54) INTERLOCK FOR DYNAMIC BONE FIXATION PLATES

(75) Inventors: LeNoir E. Zaiser, Naples, FL (US); Kevin Confoy, Naples, FL (US)

Correspondence Address: R.D. JOHNSON & ASSOCIATES, P.C., 20 PICKERING STREET, P.O.BOX 920353, NEEDHAM, MA, 02492, US

(73) Assignee: STRUCTURE MEDICAL, INC., Naples, FL (US)

(21) Appl. No.: 12/020,312

(22) Filed: Jan. 25, 2008

Related U.S. Application Data
(60) Provisional application No. 60/886,916, filed on Jan. 26, 2007.

(51) Int. Cl. A61B 17/56; A61B 17/80

(52) U.S. Cl. 606/71; 606/298

(57) ABSTRACT

Individual plate sections of a dynamic bone fixation plate can be internally interlocked to maintain the assembled plate and to limit relative motion between the sections. A dynamic bone fixation plate can include a first plate section, a second plate section, and a compressible interlock member. The first plate section includes a first joint structure and the second plate section includes a second joint structure, where the second joint structure can be dynamically mated with the first joint structure. The compressible interlock member can be disposed within the first joint structure and the second joint structure to limit relative motion of the first joint structure and the second joint structure.

### RELATED APPLICATION

This application claims the benefit of U.S. Provisional Application No. 60/886,916 entitled “Dynamic Cervical Plate”, filed on Jan. 26, 2007, the entire teachings of which are incorporated herein by reference.

### BACKGROUND

Various types of implantable devices are useful in fixing bones in the body. The structure of the device is typically dependent on the bone or bone sections being fused. One type of fixation device is a cervical plate, which are implanted to increase neck stability and to promote fusion of adjacent vertebrae following surgery to remove a diseased or damaged disc in the spine. The cervical plates are available as either static or dynamic structures.

A typical static cervical plate is a single metal piece that uses screws to attach the plate to two (or more) adjacent vertebrae. Because these plates are one piece of metal, they are relatively rigid, allowing little of no movement between the connected vertebrae.

More recently, a “dynamic” plate technology has been developed, whereby two or more individual plate sections or pieces are joined together to form the implanted cervical plate. The union between the sections allows for some movement between the individual pieces, while still providing stability and promoting bone fusion. Therefore, when it is installed, the vertebrae will have a small level of movement. Typically, dynamic plate sections are mated together using a male/female dovetail design, but other mating designs are possible.

In both cases, the cervical plate is typically made from a rigid biocompatible material, such as Titanium or stainless steel.

### SUMMARY

One challenge to constructing dynamic fixation plates is in connecting the individual parts in a manner that allows movement of the plates, but does not allow the plates to become unintentionally disassembled. In accordance with particular embodiments of the invention, individual plate sections can be internally interlocked to maintain the assembled plate and to limit relative motion between the sections.

In accordance with a particular embodiment of the invention, a dynamic bone fixation plate can include a first plate section, a second plate section, and a compressible interlock member. The first plate section includes a first joint structure and the second plate section includes a second joint structure, where the second joint structure can be dynamically mated with the first joint structure. The compressible interlock member can be disposed within the first joint structure and the second joint structure to limit relative motion of the first joint structure and the second joint structure.

More particularly, the first joint structure and the second joint structure can mate as a dovetail joint. When joined, the first and second plate sections can form a dynamic cervical plate.

The first joint structure can include a slot and the second joint structure can include a channel, the slot and channel being aligned. Furthermore, the interlock member can be disposed within the slot and channel. The first plate section can be moveable relative to the second plate section by a distance based on the dimensions of the channel. In addition, an access port can extend from the channel to the outside of the second joint structure.

The interlock member can comprise a superelastic material, which can be machined. More particularly, the material can be a Nickle-Titanium alloy, such as Nitinol materials.

In accordance with another particular embodiment, a dynamic cervical plate can comprise a first plate section, a second plate section, and a compressible interlock member. The first plate section can include a male dovetail structure and the second plate section can include a female dovetail cavity, where the male dovetail structure is slidably mated with the female dovetail cavity. The compressible interlock member is disposed within the male dovetail structure and the female dovetail cavity to limit relative motion of the male dovetail structure within the female dovetail cavity.

In accordance with another particular embodiment, a dynamic cervical plate can comprise a first plate section, a second plate section, and a superelastic interlock member. The first plate section can include a male dovetail structure and the second plate section can include a female dovetail cavity, where the male dovetail structure is slidably matable with the female dovetail cavity. The superelastic interlock member can be disposed within the male dovetail structure and the female dovetail cavity to limit relative motion of the male dovetail structure within the female dovetail cavity.

Embodiments of the invention can also include methods of manufacturing and using the described plates.

### BRIEF DESCRIPTION OF THE DRAWINGS

The foregoing and other objects, features and advantages of the invention will be apparent from the following more particular description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is an exploded perspective view of an exemplary prior art dynamic cervical plate.

FIG. 2 is an exploded perspective view of a particular dynamic cervical plate having an interlock system in accordance with the invention.

FIG. 3 is a cross-sectional view of a preassembled dovetail joint for the fixation plate assembly of FIG. 2.

FIG. 4 is a cross-sectional view of an assembled dovetail joint for the fixation plate assembly of FIG. 2.

### DETAILED DESCRIPTION

Multi-section dynamic bone fixation devices are known in the art and are commercially available from various manufacturers. In general, each manufacturer incorporates its own specific solution for mating the sections. For cervical plates, dovetail mating is typical. For illustration purposes, the concepts of the invention are described with reference to a specific dynamic cervical plate. The invention, however, is not limited to the described cervical plate its specific mating solution.

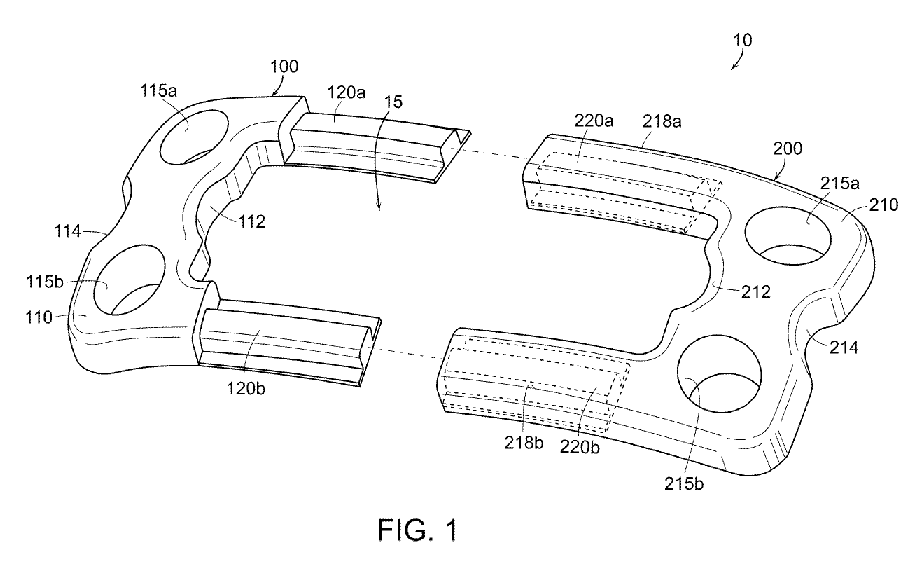

FIG. 1 is an exploded perspective view of an exemplary prior art dynamic cervical plate 10. The plate 10 includes a first section 100 and a second section 200, which, when assembled, define a window or void 15. The plate 10 is generally fabricated from a rigid biocompatible material, such as Titanium alloys.

As shown, the first section 100 includes a first main body 110 and the second section includes a second main body 210. Mounting holes 115a, 115b, 215a, 215b extend through the main body 110, 210 for receiving screws that mount the assembled plate 10 to the desired vertebrae. Also shown are interior contours 112, 212 and exterior contours 114, 214.

The plate sections 100, 200 mate using a male/female dovetail interconnect. Each section 100, 200 is generally U-shaped having leg structures 120 and 220, respectively. The leg structures 120, 220 slidably mate to provide dynamization when attached to the bone. The first section 100 includes legs 120a, 120b that are fabricated as male dovetails. The second section 200 includes legs 218a, 218b that have respective female cavities 220a, 220b dimensioned to receive the male legs 120a, 120b.

Once assembled, the plate sections 100, 200 are secured to the bone, but the plate sections 100, 200 are free to move relative to each other because the leg structures 120, 218 can slide relative to each other. The sections 100, 200 can slide apart, especially during surgery. One challenge to constructing dynamic fixation plates is in connecting the individual parts in a manner that allows movement of the plates, but does not allow the plates to become unintentionally disassembled.

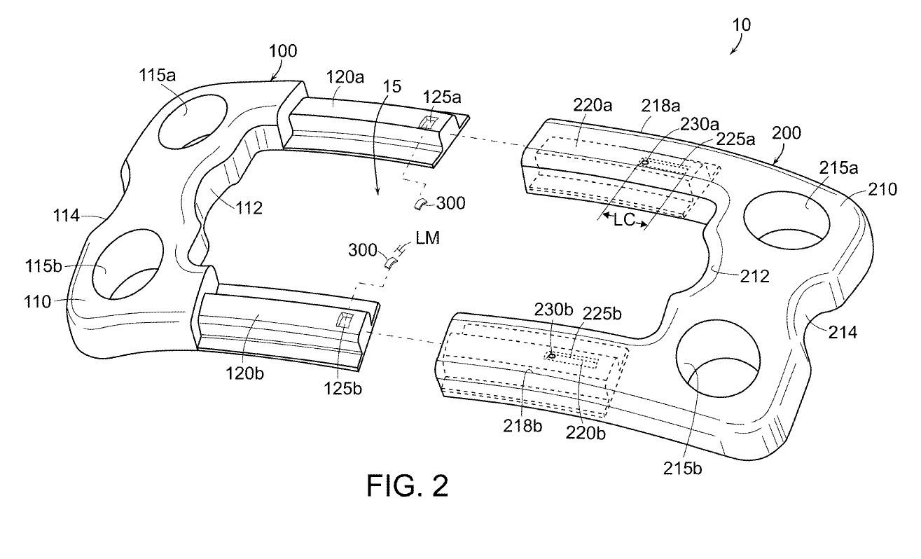

FIG. 2 is an exploded perspective view of a particular dynamic fixation plate having an interlock system in accordance with the invention. As shown, the male dovetails 120a, 120b include an interlock slot 125a, 125b. The female dovetail cavities 220a, 220b include an additional interlock channel 225a, 225b.

During assembly, a compressible interlock member 300 is seated in the interlock slots 125. The interlock member 300 is then compressed into the slot 125 and the male dovetail leg 120 is slid into the female dovetail cavity 220. As shown, the interlock member 300 is an arch shaped member resembling a miniature leaf spring.

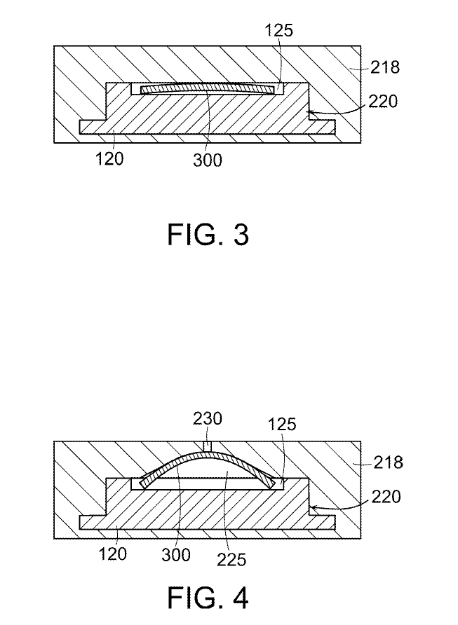

FIG. 3 is a cross-sectional view of a preassembled dovetail joint for the fixation plate assembly of FIG. 2. As shown, the interconnect slot 125 is rectangular in cross section and the male dovetail leg 120 is positioned inside the female dovetail cavity 220. The interlock member 300 is compressed within the space of the interconnect slot 125.

Returning to FIG. 2, once the interlock member 300 is registered with the interlock channel 225, the interlock member 300 expands to be received by the interlock channel 225. The interlock member 300 is then within both the interlock slot 125 and the interlock channels 225 such that the interlock member 300 is essentially incompressible in the direction of sliding. Thus, the male dovetail leg 120 cannot be slid out of the female dovetail cavity 220.

FIG. 4 is a cross-sectional view of an assembled dovetail joint for the fixation plate of FIG. 2. As shown, the interconnect channel 225 has an arch shaped cross section and the interconnect slot 125 is aligned with the interconnect channel 225. In addition, the interlock member 300 has expanded to occupy both the interconnect slot 125 and the interconnect channel 225. Note that the arch shape of the interconnect channel 225 complements the arch shape of the expanded interlock member 300, but that complementary shape for the interconnect channel 225 is not required. As shown, the interlock member 300 is under some compression.

Also shown is an access port 230, which extends from the female dovetail cavity 225 to the exterior of the female leg 218. To remove the male dovetail leg 125 from the female dovetail cavity 220, the interlock member 300 must be compressed into the interconnect slot 125 or channel 225. To that end, a tool such as pin or needle can then be inserted into the access port 230 to engage and compress the interlock member 300. In the particular embodiment of FIG. 2, the legs are separated to about their maximum extension so that the interconnect slot 125 is aligned with the access port 230. Once compressed into the interconnect slot 125, the male dovetail legs 120 are disengaged from the female leg 218 and can slid out of the female dovetail cavity 220.

As shown in FIG. 2, the amount of relative motion between the two sections 100, 200 is defined by the dimensions of the interlock slots 125 and interlock channels 225, in particular the longitudinal length LC of the interlock channels 225 minus the length LM of the interlock member 300.

In a particular embodiment, the compressible interlock member 300 is fabricated from a malleable biocompatible material. In a particular embodiment, the malleable material is a Nickle-Titanium alloy, such as Nitinol, which has shape memory and superelastic properties at body temperatures. In use, the Nitinol interlock member 300 deforms under compression, but because of the superelastic effect, the spring will return to its original shape.

More particularly, the interlock member 300 can be machined from a Nitinol bar, which can provide improved performance over similarly shaped springs that are stamped from Nitinol sheet material. In a specific embodiment, the interlock member 300 is machined from a 0.250 inch (nominal) diameter bar of SE-510 Nitinol, commercially available from Nitinol Devices and Components, Inc. of Fremont, Calif. A particular alloy bar is superelastic straight, centerless ground, with an Aƒ at about 10° C. Any Nitinol alloy having an Aƒ at between about 10° C. and 25° C. would be acceptable, with 18° C. being a target temperature. Depending on the design specifics, other Nitinol alloys, or other superelastic materials, with varying characteristics can also be used for the interlock member 300. Because the interlock member 300 can be machined, its shape is not constrained by limitations inherent in wire or sheet materials.

As shown, the interlock member 300 is an arch-shaped member similar to a miniature leaf spring. The dimensional constraints on the interlock member 300 are that it should fit within the interconnect slot 125 when compressed (such as being flattened) and that its expanded free height should be higher than the interconnect slot 125. In a particular embodiment, the expanded free height of the interlock member is at least as high as the combined heights of the interconnect slot 125 and the interconnect channel 225. Consequently, the dimensions of the interlock member 300, the interconnect slot 125 and the interconnect channel 225 are interrelated.

It should be understood that other interlock members forms can be employed with corresponding modifications to the interconnect slots and interconnect channels. While not limiting, examples of such other forms are disclosed in the incorporated provision application. The concepts of the invention are not limited to the disclosed forms, as one of ordinary skill in the art can readily appreciate other useable forms. In addition, the concepts of the invention are not limited to cervical plates and can be applied to other dynamic plate systems beyond that shown in FIGS. 1-4.

While this invention has been particularly shown and described with references to particular embodiments, it will be understood by those skilled in the art that various changes in form and details may be made to the embodiments without departing from the scope of the invention encompassed by the appended claims. For example, various features of the embodiments described and shown can be omitted or combined with each other.

What is claimed is:

1. A dynamic bone fixation plate, comprising:
a first plate section having a first joint structure;
a second plate section having a second joint structure, wherein the second joint structure is dynamically mated with the first joint structure; and
a compressible interlock member disposed within the first joint structure and the second joint structure to limit relative motion of the first joint structure and the second joint structure.

2. The plate of claim 1 wherein the first joint structure and the second joint structure mate as a dovetail joint.

3. The plate of claim 1 wherein the first joint structure includes a slot and the second joint structure includes a channel, the slot and channel being aligned.

4. The plate of claim 3 wherein the interlock member is disposed within the slot and channel.

5. The plate of claim 4 wherein the first plate section is moveable relative to the second plate section by a distance based on the dimensions of the channel.

6. The plate of claim 4 further comprising an access port extending from the channel to the outside of the second joint structure.

7. The plate of claim 1 wherein the interlock member comprises a superelastic material.

8. The plate of claim 7 wherein the material is a Nickle-Titanium alloy.

9. The plate of claim 1 wherein the first and second plate sections join to form a dynamic cervical plate.

10. A dynamic cervical plate, comprising:
a first plate section having a male dovetail structure;
a second plate section having a female dovetail cavity, wherein the male dovetail structure is slidably mated with the female dovetail cavity; and
a compressible interlock member disposed within the male dovetail structure and the female dovetail cavity to limit relative motion of the male dovetail structure within the female dovetail cavity.

11. The plate of claim 10 wherein the male dovetail structure includes a slot and the female dovetail cavity includes a channel, the slot and channel being aligned.

12. The plate of claim 11 wherein the interlock member is disposed within the slot and channel.

13. The plate of claim 12 wherein the first plate section is moveable relative to the second plate section by a distance based on the dimensions of the channel.

14. The plate of claim 12 further comprising an access port extending from the channel to the outside of the second plate section.

15. The plate of claim 10 wherein the interlock member comprises a superelastic material.

16. The plate of claim 15 wherein the material is a Nickle-Titanium alloy.

17. The plate of claim 10 wherein the interlock member is a machined member.

18. A dynamic cervical plate, comprising:
a first plate section having a male dovetail structure;
a second plate section having a female dovetail cavity, wherein the male dovetail structure is slidably mated with the female dovetail cavity; and
a superelastic interlock member disposed within the male dovetail structure and the female dovetail cavity to limit relative motion of the male dovetail structure within the female dovetail cavity.

19. The plate of claim 18 wherein the male dovetail structure includes a slot and the female dovetail cavity includes a channel, the slot and channel being aligned.

20. The plate of claim 19 wherein the interlock member is disposed within the slot and channel.

21. The plate of claim 20 wherein the first plate section is moveable relative to the second plate section by a distance based on the dimensions of the channel.

22. The plate of claim 20 further comprising an access port extending from the channel to the outside of the second plate section.

23. The plate of claim 18 wherein the superelastic interlock member comprises a Nickle-Titanium alloy.

24. The plate of claim 18 wherein the interlock member is a machined member.

25. A method of manufacturing a dynamic bone fixation plate, comprising:
fabricating a first plate section having a first joint structure;
fabricating a second plate section having a second joint structure, wherein the second joint structure is slidably matable with the first joint structure; and
fabricating a compressible interlock member disposable within the first joint structure and the second joint structure to limit relative motion of the first joint structure and the second joint structure.

26. The method of claim 25 wherein the first joint structure and the second joint structure are fabricated to mate as a dovetail joint.

27. The method of claim 25 further comprising fabricating a slot in the first joint structure and fabricating a channel in second joint structure, the slot and channel being alignable.

28. The method of claim 27 wherein fabricating the interlock member comprises dimensioning the interlock member to be disposable within the slot and channel.

29. The method of claim 28 wherein the first plate section is moveable relative to the second plate section by a distance based on the dimensions of the channel.

30. The method of claim 28 further comprising fabricating an access port extending from the channel to the outside of the second joint structure.

31. The method of claim 25 wherein the interlock member comprises a superelastic material.

32. The method of claim 31 wherein the material is a Nickle-Titanium alloy.

33. The method of claim 25 wherein the first and second plate sections are joinable to form a dynamic cervical plate.

34. A method of manufacturing a dynamic cervical plate, comprising:
fabricating a first plate section having a male dovetail structure;
fabricating a second plate section having a female dovetail cavity, wherein the male dovetail structure is slidably matable with the female dovetail cavity; and
fabricating a compressible interlock member disposable within the male dovetail structure and the female dovetail cavity to limit relative motion of the male dovetail structure within the female dovetail cavity.

35. The method of claim 34 further comprising fabricating a slot in the male dovetail structure and fabricating a channel in the female dovetail cavity, the slot and channel being alignable.

36. The method of claim 35 wherein fabricating the interlock member comprises dimensioning the interlock member to be disposable within the slot and channel.

37. The method of claim 36 wherein the first plate section is moveable relative to the second plate section by a distance based on the dimensions of the channel.

38. The method of claim 36 further comprising fabricating an access port extending from the channel to the outside of the second plate section.

39. The method of claim 34 wherein the interlock member is fabricated from a superelastic material.

40. The method of claim 39 wherein the material is a Nickle-Titanium alloy.

41. The method of claim 40 wherein fabricating the interlock member comprises machining the Nickel-Titanium alloy.

42. A method for fabricating a dynamic cervical plate, comprising:
fabricating a first plate section having a male dovetail structure;
fabricating a second plate section having a female dovetail cavity, wherein the male dovetail structure is slidably matable with the female dovetail cavity; and
fabricating a superelastic interlock member disposable within the male dovetail structure and the female dovetail cavity to limit relative motion of the male dovetail structure within the female dovetail cavity.

43. The method of claim 42 further comprising fabricating a slot in the male dovetail structure and fabricating a channel in the female dovetail cavity, the slot and channel being alignable.

44. The method of claim 43 wherein the interlock member is dimensioned to be disposable within the slot and channel.

45. The method of claim 44 wherein the first plate section is moveable relative to the second plate section by a distance based on the dimensions of the channel.

46. The method of claim 44 further comprising fabricating an access port extending from the channel to the outside of the second plate section.

47. The method of claim 42 wherein the superelastic interlock member comprises a Nickle-Titanium alloy.

48. The method of claim 42 wherein fabricating the interlock member comprises machining a superelastic material.
